(19) United States
(12) Patent Application Publication
(10) Pub. No.: US 2022/0168073 A1
(43) Pub. Date: Jun. 2, 2022

(54) IMPLANT SET AND METHOD FOR PREPARING FOR INSERTION OF AN IMPLANT

(71) Applicant: KARL LEIBINGER MEDIZINTECHNIK GMBH & CO. KG, Mühlheim (DE)

(72) Inventors: Adem AKSU, Villingen-Schwenningen (DE); Frank REINAUER, Emmingen-Liptingen (DE); Tobias WOLFRAM, Dreieich (DE)

(21) Appl. No.: 17/442,774

(22) PCT Filed: Apr. 6, 2020
(86) PCT No.: PCT/EP2020/059755

§ 371 Date: Sep. 24, 2021

(30) Foreign Application Priority Data
Apr. 18, 2019 (DE) 10 2019 110 385.1

(51) Int. Cl. A61C 8/02; A61C 8/00; A61L 27/12

(52) U.S. Cl. CPC A61C 8/0006; A61L 2430/12; A61L 27/12; A61C 8/0089

(57) ABSTRACT

The disclosure relates to an implant set including a patient-specific implant, including a transport device, which is matched to an outer contour of the implant and in which the implant is received, and includes a tool for filling the implant with bone chips and/or for compressing the bone chips, the implant having an inner contour to which an outer contour of at least one end of the tool is matched. The disclosure further relates to a method for preparing for insertion of the implant by means of the implant set.

The present disclosure relates to an implant set. Furthermore, the invention relates to a method for preparing insertion of an implant.

In order to fill bone cavities and/or for bone regeneration, patient-specific, custom-made implants are used. These implants have a three-dimensional complex shape and differ significantly from each other depending on the patient and/or implantation site. At the present time, there are no auxiliary means, tools or structured devices that are used to fill these implants with bone chips and/or other tissue structures, such as cartilage or osteochondral tissue structures, or to fix these implants to the bone in a supportive manner.

It is therefore the object of the invention to avoid or at least reduce the disadvantages of the prior art. In particular, an implant set and a method for preparing insertion of the implant are to be proposed, which enable particularly efficient and optimized preparation and/or handling of the implant.

The object of the invention is solved by an implant set having the features of patent claim 1 and by a method having the features of patent claim 10. Advantageous embodiments are claimed in the dependent claims and are explained in more detail below.

More specifically, according to the invention, an implant set is proposed. The implant set has a patient-specific implant, in particular a jaw clamp or a jaw plate for an upper jaw. Furthermore, the implant set comprises a transport device which is adapted to an outer contour of the implant and in which the implant is inserted/placed. An inner contour preferably corresponds to the outer contour, at least to part of the outer contour, of the implant on several sides, for example in a bowl-like manner, and/or in a flat and/or flush/fitting manner. Furthermore, the implant set has, for example, a tappet-like tool for filling the implant with bone chips and for pressing the bone chips.

In addition, the implant set may also be constructed in such a way that soft tissues, e.g. cartilage, collagen materials or bone-cartilage mixtures are used instead of bone chips, and tools are used for tissue-conserving compaction of these materials. The implant has an inner contour to which an outer contour of at least an end of the tool is adapted. In other words, the shape of the tool, in particular at its end, is adapted to the shape of the implant such that its shape corresponds at least in part to the shape of the implant, in particular to the inner contour, such as a through hole in the implant. The tool may serve as an interface with the function of fixing the implant with minimal time and/or force required, and/or with the function of filling the implant with bone chips and/or compressing these bone chips in the implant. The tool may also function as an interface with or without the function for a specific energy transfer.

It is particularly preferred if the outer contour of the implant lies against the inner contour of the transport device in such a way that bone chips in the inner contour of the implant can be pressed between the tool and the transport device (in the inner contour of the implant). In other words, according to a particularly preferred embodiment, the implant may have (as inner contour) a (first) opening, such as a through hole, for example open towards the outer contour, wherein the opening, when the implant lies/is inserted in the transport device, is closed by the inner contour of the transport device, preferably flush following the outer contour of the implant. This allows bone chips to be pressed in the opening without falling out during pressing, while also being in direct surface contact with the bone at the implant site. In particular, the bone chips may be filled through an entry opening on one side of the through hole in the implant and may be pressed by the tool starting from the entry opening. The fact that the first opening is closed by the transport device prevents the bone chips from falling out during pressing, but at the same time they can be pressed in such a way that, when removed from the transport device, a flush outer contour/outer surface of the implant is formed by the bone chips pressed in the through hole.

This has the advantage that, for example, bone chips may be filled into the implant and can be uniformly compacted by the tool when the outer shape of the tool corresponds to the contour of a recess in the implant in which the bone chips are located. By filling with bone chips, the strength of the implant can be increased in a simple way. Thus, the tool may be used in a versatile manner and for multiple applications, so that a reduced number of tools is required. In particular, an outer contour of the tool is slightly smaller than an inner contour of the implant, since the tool can thus be inserted into the implant particularly easily without any risk of damaging the implant.

According to a preferred embodiment, the tool may have at least two ends, which are configured as rods. Thus, the two ends may be used for different parts of the inner contour of the implant. A rod-shaped configuration of the tool enables the tool to also engage in deep recesses in the implant. Preferably, the tool has, at least in sections, a constant cross-section over its extension in the longitudinal direction.

According to an advantageous further development of the embodiment, the ends of the tool may have different cross-sectional geometries. For example, a portion of the tool may have a round cross-section, in particular a circular cross-section. A portion of the tool may have an oval cross-section. Round cross-sections are particularly suitable for engaging holes, such as bores, in particular dental bores/dental holes in the implant, for example to compress and/or compact bone chips filled therein. It is also possible for a portion to have an angular cross-section, for example a triangular, rectangular or square cross-section. Such cross-sectional shapes are suitable for compressing the bone chips in, for example, a slit-shaped indentation in the implant.

Alternatively, the tool may have a portion with a tool engagement geometry. For example, the tool engagement geometry may have a cross-slot, Torx, hexagonal, or Allen drive profile. Thus, the tool can be used to apply an insertion torque or an extraction torque.

The tool may also have a portion with a spatula, spoon or scoop geometry. Such a geometry can advantageously be used to fill the bone chips or other, softer tissue structures, e.g. cartilage, from a container into the implant. If the portion has a rectangular cross-section, it is suitable both as a scoop and as a compactor for slit-shaped indentations.

According to a preferred embodiment, the tool may have a gripping region, which allows easy gripping and/or handling of the tool. In particular, the gripping region may have a circular profile that is flattened on both sides or on all sides or may have a rectangular cross-section, preferably with rounded edges and/or slightly spherical side surfaces, which enables a particularly good grip in one hand in a simple manner.

In a preferred embodiment, a front side/front surface of the tool, which may also be referred to as a distal surface, may be convex/spherical, concave or planar/flat. A planar or slightly spherical front surface has proven to be particularly suitable, since this allows the bone chips to be pressed particularly evenly.

The tool may be constructed of polyamide (PA), polyetheretherketone (PEEK), polyoxymethylene (POM), polyphenylene sulfone (PPSU), stainless steel, titanium, ceramics, or a combination of the above materials. Preferably, the tool is constructed of polyamide. The tool may have a coating and/or surface modification. In addition, the tool may preferably be sterile.

According to a preferred embodiment, the transport device may have a tool receptacle prepared for holding the tool in a material-fitting, form-fitting and/or force-fitting manner. Preferably, the tool may be detachably, i.e. reattachably, received in the tool receptacle. Particularly preferably, the tool may be detached from the tool receptacle without the use of a tool, i.e., without the use of another tool. According to an advantageous further development of the embodiment, the tool receptacle may form a predetermined breaking point between the tool and the transport device and/or be adapted, for example, as an attachment to the outer contour of the tool. This ensures that the required tool is ready to use.

According to an advantageous embodiment, the transport device may have a main body and a closure body for holding and/or contacting the implant on multiple sides. In this way, the implant can be securely held between the main body and the closure body, in particular in order to fill it with bone chips or cartilage fragments.

According to a particularly preferred further development, the closure body may be displaceable, in particular guided in a displaceable manner, relative to the main body, preferably linearly. Alternatively, the closure body may be pivotable or foldable relative to the main body. In particular, the closure body is displaceable relative to the main body between a first position in which the closure body abuts a portion of the main body such that the implant is enclosed, preferably completely or on multiple sides, and a second position in which the closure body is spaced from the portion of the main body. In particular, it is preferred if the displacement of the closure body is guided by a rail in a form-fitting manner. For example, the rail is formed by a groove formed on the main body (or on the closure body) and by a corresponding projection formed on the closure body (or on the main body). The groove may, for example, have the shape of a dovetail.

According to the embodiment, the transport device has a receptacle for the implant, wherein the receptacle is formed by the closure body and the main body. The receptacle may, for example, have a bowl-like shape. For example, the receptacle may have a bell-shaped or round cross-section/a bell-shaped inner contour shape.

In an advantageous further development, the closure body (or main body) may have pins and the main body (or the closure body) may have corresponding holes, wherein the pins engage in the holes in the first position. As a result, the transport device has a particularly high stability in the first position. In particular, it is advantageous if the pins (or spikes or bolts) extend in a direction parallel to the direction along which the closure body is displaceable relative to the main body. In addition, it is preferred if an insertion direction of the tool, in which the tool can be inserted into the tool receptacle, is oriented parallel to the direction along which the closure body is displaceable relative to the main body. In this way, a particularly compact implant set can be provided.

The transport device may have a gripping lug that protrudes from the closure body, for example. This allows the transport device to be easily gripped and/or transported.

The transport device may be constructed of polyamide (PA), polyetheretherketone (PEEK), polyoxymethylene (POM), polyphenylene sulfone (PPSU), stainless steel, titanium, ceramics or a combination of the above materials. Preferably, the transport device is constructed of polyamide. The transport device may have a coating and/or a surface modification. In addition, the transport device may preferably be sterile.

According to a preferred embodiment, the implant may be configured as a jaw clamp or a jaw plate. In particular with implants configured as a jaw plate, it has been shown that filling with bone chips is particularly beneficial.

In an advantageous embodiment, the implant may have, at least in sections, a grid-like or rhombic or honeycomb structure or a pore structure. This enables wedging of the bone chips in the implant. For example, the implant may have the shape of a shell, for example with a U-shaped or a V-shaped profile.

According to a preferred embodiment, the implant may have at least one recess, in particular a through hole, which serves as a dental bore. For example, the implant has two through holes and/or a bar-like indentation which may be filled with bone chips.

The implant may be constructed of a resorbable bone replacement material. The implant is preferably constructed of hydroxylapatite (HA), α-tricalcium phosphate (α-TCP), β-tricalcium phosphate (β-TCP), biphasic calcium phosphate (BCP), magnesium (Mg), MgCaZn, Bioglass, molybdenum (Mo), or a combination of the materials. In addition, the implant may preferably be sterile.

According to the invention, a method for preparing insertion of the implant via the implant set according to the invention is also proposed. In a preliminary step, the transport device may be opened so that the implant can be inserted. In a first step, the implant is inserted into the transport device. Then, the transport device can be closed. Then, in a second step, bone chips are filled into the implant with the tool, in particular with the first tool portion. Then, in a third step, the bone chips are pressed into the implant with the tool, in particular with the second tool portion. For this purpose, the tool is pressed into the through hole or into the recess/indentation in the implant. The transport device can then be opened and the implant can be removed from the transport device. Finally, the implant is fixed to a skull bone, for example the upper jaw. The tool may also be used to hold and/or fix the implant and/or to press the bone chips to the upper jaw.

### BRIEF DESCRIPTION OF THE FIGURES

The invention is explained below with the aid of drawings. The following is shown:

FIG. 1 shows a perspective view of a transport device and of a tool of an implant set according to the invention,

FIG. 2 shows a perspective view of the implant set with an implant, the transport device, and the tool,

FIG. 3 shows a perspective view of the tool, FIGS. 4 and 5 show a perspective view of using the tool, and FIGS. 6 to 9 show perspective views of the implant set and steps of a method for preparing the implant.

The figures are merely schematic in nature and are intended solely for the purpose of understanding the invention. Identical elements are referred to by the same reference signs.

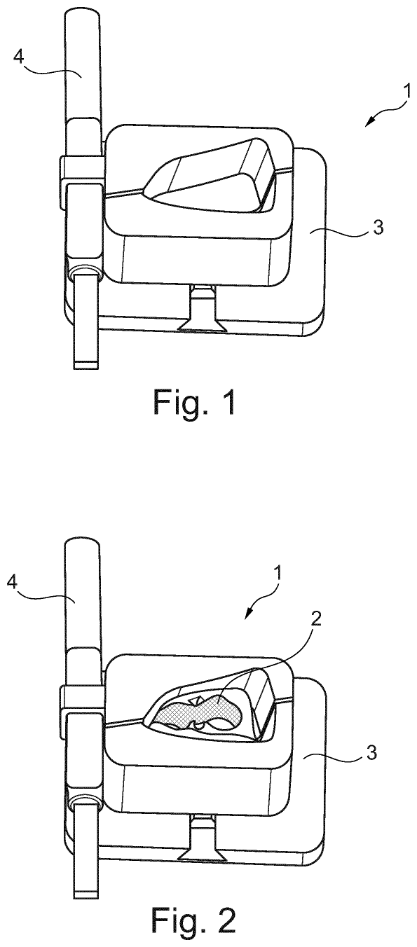

FIGS. 1 and 2 show perspective views of an implant set 1 according to the invention. The implant set 1 has a patient-specific implant 2. The implant 2 is configured as an individual implant made of bone replacement material. The implant 2 is used in particular as a resorbable bone-cavity filler and/or for bone regeneration. The implant 2 is not shown in FIG. 1. The implant set 1 has a transport device 3. The transport device 3 is adapted to a shape, in particular to an outer contour, of the implant 2. The transport device 3 is used for storing, transporting, and/or filling the implant 2 with bone chips. The implant 2 is housed in the transport device 3 (compare FIG. 2). The implant set 1 also has a tool 4. The tool 4 may be used for filling the implant 2, for example with bone chips. The tool 4 may also be used for compressing material, in particular bone chips, preferably in the implant 2. The tool 4 has at least one end 5, 6, which is adapted to the shape of the implant 2, in particular to an inner contour of the implant 2.

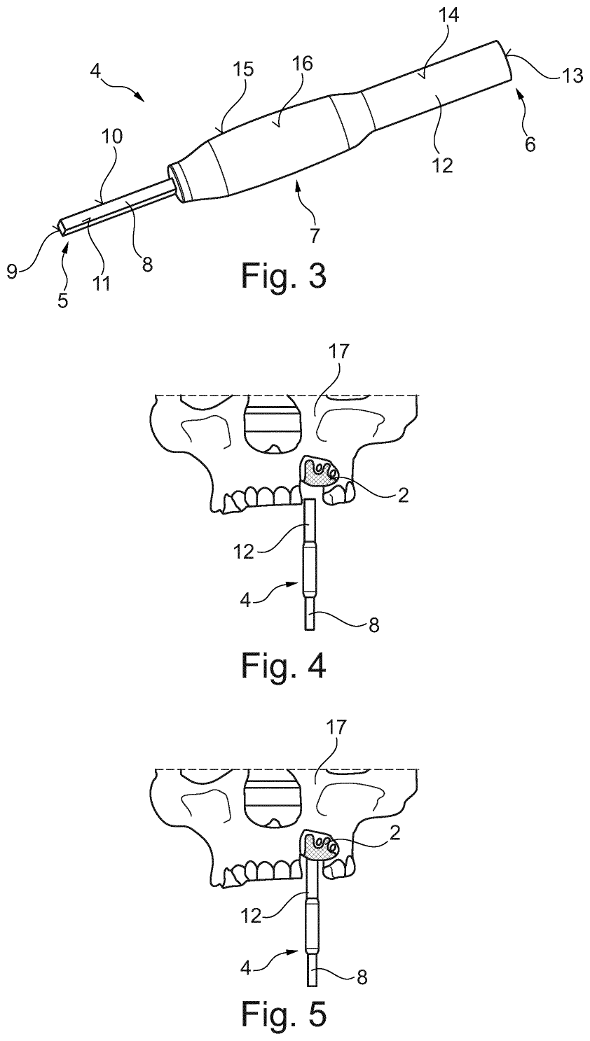

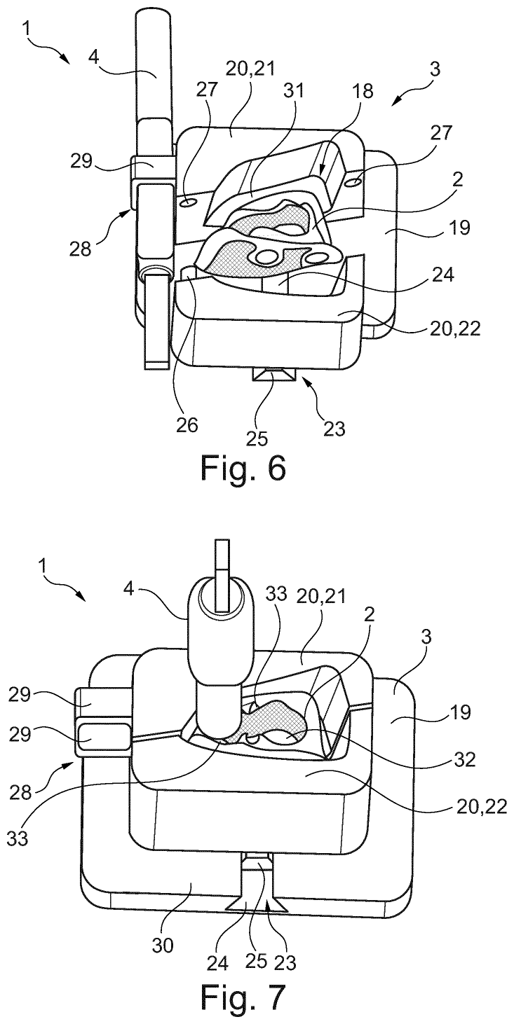

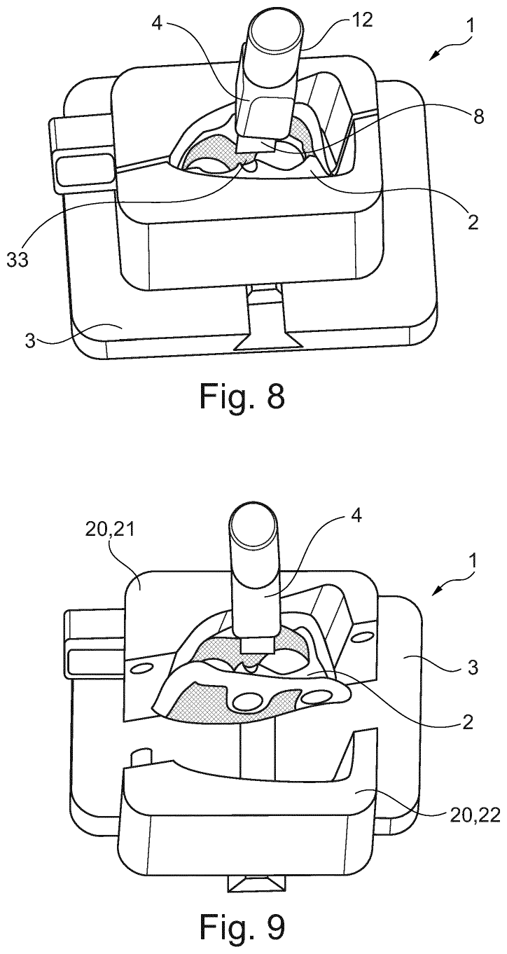

FIG. 3 shows a perspective view of the tool 4. The tool 4 is tappet-like or rod-shaped. This means that the tool 4 has a substantially greater extension along its longitudinal direction than along its transverse direction, which is transverse to the longitudinal direction. For example, the extension in the longitudinal direction may be at least twice as large as the extension in the transverse direction. Alternatively, the tool may have, for example, the shape of a cross key, although this is not shown. In the embodiment shown, the tool 4 has a first end 5 and a second end 6 opposite the first end 5. The tool 4 has a gripping region 7, which is arranged between the two ends 5. The tool 4 may be constructed from one or more, for example two, materials. Polyamide (PA), polyetheretherketone (PEEK), polyoxymethylene (POM), polyphenylene sulfone (PPSU), stainless steel, titanium, ceramics or a combination of the above materials have been found to be particularly suitable materials. Preferably, the tool 4 is constructed of ceramics.

A first tool portion 8 arranged at the first end 5 has a substantially rectangular cross-section. The cross-section of the first tool portion 8 is constant over its extension in the longitudinal direction. The first tool portion 8 has rounded edges. A front surface 9 of the first tool portion 8 is planar or flat. However, the front surface 9 may also be concave or convex, although this is not shown. Due to the rectangular cross-section, the first tool portion 8 has two wide side surfaces, hereinafter referred to as first side surfaces 10, and two narrow side surfaces, hereinafter referred to as second side surfaces 11. One of the first side surfaces 10 may be used, for example, as a scoop with which the bone chips can be picked up and can be filled into the implant 2. For example, the bone chips may be taken from a body part, such as a pelvis. Preferably, the removed bone chips are crushed in a container, such as a stainless steel cup, for example with scissors, and are removed from the container by the tool 4, in particular by the first side surface 10 of the first tool portion 8.

A second tool portion 12 arranged at the second end 6 has a substantially round, in particular circular, cross-section. The cross-section of the second tool portion 12 is constant over its extension in the longitudinal direction. A front surface 13 of the second tool portion 12 is planar or flat. However, the front surface 13 may also be concave or convex, although this is not shown. The second tool portion 12 has an external peripheral surface 14. The front surface 13 of the second tool portion 12 is used in particular for compacting or pressing the bone chips in the implant 2. Preferably, an outer contour of the second tool portion 12 corresponds at least in sections to an inner contour of the implant 2, in particular of dental holes of the implant 2, which are described in more detail below, which enables particularly uniform compaction.

The gripping region 7 has a substantially rectangular, for example square, cross-section. The gripping region 7 has rounded edges. The gripping region 7 has four side surfaces 15. In the embodiment shown, two opposite first side surfaces 15 are slightly convex, i.e., spherical towards the outside. In particular, the first side surfaces 15 may be spherical in the longitudinal direction, so that the cross-section is largest in the center of the gripping region 8 and becomes smaller in the longitudinal direction towards the outside. However, the first side surfaces 15 may also be planar or concave, although this is not shown. Two opposite second side surfaces 16 are planar in the embodiment shown, but may also be convex or concave, even if this is not shown.

FIGS. 4 and 5 show a possible use of the tool 4. In the embodiment shown, the implant 2 is attached to a skull bone, in particular an upper jaw 17. The tool 4 may be used to hold and/or fix the implant 2. The tool 4 may also be used for additional compression of the bone chips in the implant 2 attached to the upper jaw 17, in that the tool 4, in particular the second tool portion 12, compresses the bone chips in the direction towards the upper jaw 17. However, the tool 4 may mainly be used for preparing the insertion of the implant 2, which is described in more detail below.

Features of the implant 2 and of the transport device 3 are described in more detail with reference to FIGS. 6 to 9. A method according to the invention for preparing an insertion of the implant 2 is also described.

The transport device 3 has a receptacle 18 for the implant 2. In the embodiment shown, the receptacle 18 is designed as a recess in the transport device 3 into which the implant 2 can be inserted, in particular with a precise fit and/or shape. In the embodiment shown, the receptacle 18 is open to one side, in particular upward in a vertical direction. The transport device 3 also has, for example, a plate-shaped bottom 19. The receptacle 18 is formed by a bowl 20.

The bowl 20 has a multi-part structure. The bowl 20 has a first bowl portion 21 that forms a part of the bowl 20, for example approximately a bowl half. The first bowl portion 21 is fixedly connected to the bottom 19. Alternatively, the first bowl portion 21 may be movable relative to the bottom 19. The first bowl portion 21 may be integrally or monolithically formed with the bottom 19. However, the first bowl portion 21 may also be formed as a component separate from the bottom 19 and is attached to the bottom 19. The first bowl portion 21 and the bottom 19 form a main body of the transport device 3. The bowl 20 comprises a second bowl portion 22, which forms a part of the bowl 20, for example a (different) bowl half. The first bowl portion 21 is formed separately from the second bowl portion 22. The second bowl portion 22 is movable, in particular displaceable, relative to the first bowl portion 21. Alternatively, the second bowl portion 22 may also be pivotable, rotatable or foldable relative to the first bowl portion 21.

The bowl 20 may be moved to an open position and to a closed position. In the open position, shown in FIG. 6, the two bowl portions 21, 22 are spaced apart. In particular, the two bowl portions 21, 22 are spaced apart from each other to such an extent that the implant 2 can be pushed or inserted into the receptacle 18 through the distance. The distance is thus preferably greater than a maximum width of the implant 2. In the closed position shown in FIG. 7, the two bowl portions 21, 22 lie against each other so that the receptacle 18 has a closed contour. The contour of the receptacle 18 corresponds in particular to an outer contour of the implant 2. In the closed position, the implant 2 cannot (or should not) be inserted into or removed from the receptacle 18.

For opening or closing the bowl 20, the second bowl portion 22, which forms a closure body, may be displaced relative to the first bowl portion 21. A rail 23 is formed between the bottom 19 and the second bowl portion 22 to guide the displacement. The rail 23 is formed by a groove 24 in the bottom 19 and by a projection 25 on the second bowl portion 22. The projection 25 is arranged close to the bottom, i.e. on a bottom-facing (lower) side of the second bowl portion 22. Alternatively, the groove may be formed on the second bowl portion 2 and the projection may be formed on the bottom 19. A cross-section of the groove 24 corresponds to a cross-section of the projection 25. In the embodiment shown, the cross-section has a dovetail shape. Thus, the cross-section tapers in the direction toward the second bowl portion 22 so that the second bowl portion 22 cannot be removed in a direction perpendicular to the bottom. The groove 24 extends in a direction referred to hereinafter as a displacement direction. The groove 24 has an opening towards an outer side of the transport device 3. The second bowl portion 22 can thus be displaced along the displacement direction in the groove 24 for opening and closing the bowl 20. Via the rail 23, the second bowl portion 22 is attached to the bottom 19 in a form-fitting manner (in a vertical direction), but in a displaceable manner (linearly) along the displacement direction.

Two pins 26 are formed on the second bowl portion 22, which protrude in the direction of the first bowl portion 21. Only one pin 26 may be provided as well. More than two pins, for example 3 or 4 pins, may also be provided. The pins 26 extend along the displacement direction. A corresponding number of holes 27 are formed in the first bowl portion 21. The holes 27 are adapted to the diameter of the pins 26. The holes 27 are aligned with the pins 26 in the displacement direction so that the pins 26 engage the holes 27 upon closing the bowl 20. Preferably, the pins 26 and the holes 27 form a force-fit connection that increases the stability of the transport device 3 and/or prevents unintentional opening. Alternatively, the pins may be formed on the first bowl portion 21 and the holes may be formed on the second bowl portion 22, although this is not shown.

The transport device 3 has a tool receptacle 28. The tool 4 may be held in the tool receptacle in a material-fitting, form-fitting, and/or force-locking manner. In the embodiment shown, the tool receptacle 28 is configured as an attachment. The attachment is formed by two holding arms 29, for example a lower holding arm and an upper holding arm, between which the tool 4 can be inserted. The holding arms 29 are matched to the outer contour of the tool 4, for example to the contour of the gripping region 7. The tool 4 may be inserted into the attachment in the displacement direction. The tool 4 is detachably mounted in the tool receptacle 28. The tool 4 may therefore be removed from the tool receptacle 28, in particular without tools, and may be reattached in the tool receptacle 28. Alternatively, the tool 4 may be fastened to the tool receptacle 28 in a material-fitting manner by a predetermined breaking point, although this is not shown.

A gripping lug 30 is formed by the bottom 19. The bottom 19 protrudes from the bowl 20 so that the transport device 3 may be gripped at the bottom 19. For example, the gripping lug 30 may protrude to opposite sides or on all sides of the bowl 20.

In other words, the transport device 3, in particular an inner contour shape of the receptacle 18, is formed to the shape, in particular an outer contour shape, of the implant 2. The inner contour shape of the receptacle 18 may have, for example, a bell shape or a substantially triangular or rectangular shape. One side of the implant 2 rests on a bottom surface 31 of the receptacle 18, in particular in a flush manner. The bottom surface 31 is formed in part by the first bowl portion 21 and in part by the second bowl portion 22.

The transport device 3 may be constructed from one or more, for example two, materials. Polyamide (PA), polyetheretherketone (PEEK), polyoxymethylene (POM), polyphenylene sulfone (PPSU), stainless steel, titanium, ceramics or a combination of the aforementioned materials have proven to be particularly suitable materials. Preferably, the transport device 3 is constructed of ceramics.

The implant 2 is configured as an individual implant and thus varies in design depending on the application and patient. In the embodiment shown, the implant 2 is U-shaped or V-shaped. The shape of the implant 2 may also be described as a shell. Two through holes 32 are formed in the implant 2, which may also be described as dental holes or dental bores. Only one through hole may be formed in the implant as well. Alternatively, more than two through holes, for example 3, 4, 5 or 6 through holes, may be formed. The through holes 32 extend in a direction that corresponds to a vertical direction when inserted into the transport device 3. The through holes 32 are connected via a web-shaped indentation 33 formed in the implant 2. The implant 2 has a lattice structure/grid structure, which is also referred to as a pore structure or a honeycomb structure.

When the implant 2 is inserted in the receptacle 18, the implant 2, in particular the through holes 32, may be filled with bone chips using the tool 4. For this purpose, the bone chips are filled, for example scooped, into the through holes 32 with the first tool portion 8 of the tool 4. The second tool portion 12, whose outer contour corresponds to the shape of the through holes 32, is used to compact the bone chips. For this purpose, the front side 13 of the second tool portion 12 is inserted into the through holes 32 and is pressed against the bottom surface 31 of the transport device 3 (compare FIG. 7). The bone chips form a lump due to the consistency of the bone chips, the different shapes of the individual bone chips, any blood added and the grid structure of the implant 2. Therefore, the bone chips remain attached to the implant 2 even when the implant 2 is removed from the transport device 3.

FIG. 8 shows that the indentation 33 in the implant 2 is filled with the bone chips by the tool 4. The shape of the first tool portion 8 corresponds to the shape of the indentation 33. The bone chips are compacted and/or pressed by pressing the tool 4 into the indentation 33.

In order to be able to implant the implant 2, the transport device 3, in particular the bowl 20, is opened. The implant 2, which has been filled with bone chips, can then be removed from the transport device 3 (compare FIG. 9).

The implant 2 is composed of bone replacement materials such as hydroxylapatite (HA), α-tricalcium phosphate (α-TCP), β-tricalcium phosphate (β-TCP), biphasic calcium phosphate (BCP), magnesium (Mg), MgCaZn, Bioglass, molybdenum (Mo), or a combination of the materials.

We claim:

1. An implant set, comprising
a patient-specific implant, the implant having an outer contour and an inner contour,
a transport device, the transport device having an inner contour which is adapted to the outer contour of the implant and in which the implant is housed, and
a tool for filling the implant with bone chips and/or tissue structures and/or for pressing the bone chips, the tool having at least one end with an outer contour,
wherein the outer contour of the at least one end of the tool is adapted to the inner contour of the implant, and
wherein the outer contour of the implant lies against the inner contour of the transport device in such a way that bone chips in the inner contour of the implant can be pressed between the tool and the transport device.

2. The implant set according to claim 1, wherein the tool has at least two ends which are configured as rods.

3. The implant set according to claim 2, wherein the ends of the tool have different cross-sectional geometries.

4. The implant set according to claim 1, wherein a first portion of the tool has a circular cross-section.

5. The implant set according to claim 1, wherein the transport device has a tool receptacle prepared for holding the tool in a material-fitting, form-fitting, and/or force-fitting manner.

6. The implant set according to claim 5, wherein the tool receptacle forms a predetermined breaking point between the tool and the transport device.

7. The implant set according to claim 1, wherein the transport device has a main body and a closure body for holding the implant on multiple sides.

8. The implant set according to claim 7, wherein the closure body is displaceable relative to the main body between a first position in which the closure body abuts a portion of the main body such that the implant is enclosed, and a second position in which the closure body is spaced from the portion of the main body.

9. The implant set according to claim 1, wherein the implant has, at least in sections, a grid-like or rhombic or honeycomb structure or a pore structure.

10. The implant set according to claim 1, wherein the implant has a recess that can be filled with bone chips.

11. Method for preparing insertion of an implant via an implant set according to claim 1, wherein in a first step, the implant is inserted into the transport device, wherein thereafter in a second step, bone chips are filled into the implant with the tool, and wherein then in a third step, the bone chips are pressed into the implant with the tool between the tool and the transport device.

12. The implant set according to claim 1, wherein a second portion of the tool has a rectangular cross-section.

13. The implant set according to claim 5, wherein the tool receptacle is adapted to the outer contour of the tool.

14. The implant set according to claim 1, wherein the transport device has a main body and a closure body for contacting the implant on multiple sides.

15. The implant set according to claim 1, wherein the implant has a through hole that can be filled with bone chips.
